(19) United States
(12) Patent Application Publication
(10) Pub. No.: US 2021/0033515 A1
(43) Pub. Date: Feb. 4, 2021

(54) METHOD FOR QUANTIFYING POROUS MEDIA BY MEANS OF ANALYTICAL PARTICLES AND USES THEREOF

(71) Applicant: Patentpool Innovations Management GmbH, München (DE)

(72) Inventor: Thomas SCHIMMEL, Karlsruhe (DE)

(21) Appl. No.: 16/968,289

(22) PCT Filed: Mar. 8, 2019
(86) PCT No.: PCT/EP2019/055842

§ 371 Date: Aug. 7, 2020

(30) Foreign Application Priority Data
Mar. 8, 2018 (DE) 10 2018 105 394.0

(51) Int. Cl. G01N 15/08; G01N 33/24

(52) U.S. Cl. CPC G01N 15/082; G01N 15/0806; G01N 33/24

(57) ABSTRACT

The invention relates to a method for quantifying porous media and to the analytical particles specially designed therefor and to the use thereof, for example in order to determine the water permeability of rocks as a prerequisite for the development of criteria for ground water movement or the material characterization of porous materials or rock layers or for monitoring chemical, biological and/or biotechnological reactors, water tanks, water reservoirs and water line systems or in medical in-vivo methods.

### CROSS-REFERENCE

This application is a section 371 U.S. National phase of PCT/EP2019/055842, filed Mar. 8, 2019 which claims priority from German Patent Application No. 10 2018 105 394.0, filed Mar. 8, 2018, both which are incorporated by reference in its entirety.

### FIELD OF INVENTION

The invention relates to a method for quantifying porous media as well as the specially designed analysis particles (hereinafter also referred to as “particles”) and their applications, for example for determining the water permeability of rocks as a prerequisite for the development of criteria for groundwater movement or material characterization of porous materials or rock strata or for monitoring chemical, biological and/or biotechnological reactors, or in medical in-vivo methods.

### BACKGROUND OF THE INVENTION

Tracer systems in hydrology are known from the prior art. The basis of hydrogeology, is for the definition according to DIN 4049-1 and/or DIN 4039-3, is the knowledge of the structure of the subsurface. This is based on geological maps, drilling results and three-dimensional models of the geological subsurface. Tools and methods for the analysis and monitoring of the groundwater are remote sensing, geophysical methods, drilling, groundwater measuring points, pumping tests, laboratory tests and water chemical analysis. Groundwater in cracked rock or porous materials can flow in a laminar but also turbulent manner.

Since the flow paths and their properties in rock mass or porous materials are rarely fully known, it is difficult to estimate their share in the total groundwater flow. Overall, however, the influence of turbulent flow on the hydraulic permeability and the flow velocity is considered to be little. Therefore, fractured aquifers are mostly described with the continuum model in a simplified way and the movement as exclusively laminar. In the hydraulic methods, a known signal (positive or negative pressure change) acts on a mostly unknown system, called the aquifer, for example with a long-term pump test.

The reaction of the system (pressure drop, pressure rise or water level drop, water level rise) is recorded during the test per unit of time. After the signal end, the return of the system to its equilibrium state can be measured as well. In long-term pumping or injection tests, the changes in water level or pressure communicated to the aquifer are monitored over a longer period of time. According to STOBER (1993), statements about the flow behavior or the aquifer model can only be made in long-term pumping tests. For all other hydraulic tests, the evaluation model is implicitly specified.

Packer test by packer (borehole), collective term for hydraulic tests in a borehole or well, which in the single packer test are carried out in the section located above or below the packer and hydraulically separated, in the double packer test also in sections in the stretch between two packers. Single packer tests are carried out in partially open boreholes that are cased to below the water level. A thinner casing leads through the packer, through which filling tests, slug tests or oscillatory tests valid for the lower borehole section can be carried out to determine the hydraulic parameters of the aquifer. Double packer tests are carried out in the uncased borehole, the section between the two packers can be tested using the tests listed above.

The reliability of the results depends on the tightness of the packers, the nature of the borehole wall and the surrounding rock mass, or may be reduced due to seepage and/or lateral seepage in the rock mass. For rock-mechanical analyses, water pressure tests via single or double packered borehole sections are used, in which water is pressed in under different pressures to determine the kf value of solid rock, e.g. in the underground of dams (Lexicon der Geowissenschaften, Spektrum Verlag). The permeability K is used in geotechnical engineering to quantify the permeability of soil and rock to liquids or gases (e.g. groundwater, petroleum or natural gas). The permeability coefficient kf value explained here as well is very closely connected to it. The permeability coefficient (or the hydraulic conductivity) also quantifies the permeability of soil or rock, however, the density and viscosity of the fluid are also considered here (Bernward Hölting, Wilhelm G. Coldewey: Hydrogeologie: Einführung in die Allgemeine and Angewandte Hydrogeologie. 6. Auflage. Elsevier Spektrum Akademischer Verlag, Munich 2005).

Tracer methods are also used in hydrology with or without a combination of pumping tests. A tracer is an artificial or natural substance (tracer substance) which, after being introduced into a hydrological system, enables or facilitates a wide variety of analyses.

Electrolytic tracer (NaCl, MgCl₂): The tracer substance is put into an upstream gauging station (measuring point) or sinkhole (sinkage section). Resistance measurements are carried out in the well. In doing so, the increase in the ion concentration of groundwater ingress is measured over time. It is also possible to measure the dilution at the input gauging station.

Spore drift, microparticles: Lycopodium spores are colored with up to five different colors. Thus, different sinkholes with differently colored spores can be used simultaneously used as input points. Due to their density of 1.1, the spores remain floating for a long time. Their size is approximately 33 micrometers [μm]. The spores are caught using a plankton network, which usually has to be emptied daily. The evaluation is carried out microscopically by counting. Microparticles such as polystyrene beads are available in sizes from 0.05 to 90 micrometers [μm]. They are colored with fluorescent colors and can be used like colored spores. Aim of the analysis: The advantages of the method are that it does not affect the water quality or the appearance of the water, and the possibility of being able to distinguish different sinkholes due to the spore colors.

The disadvantages are that only qualitative statements about the flow paths and the flow velocities can be made. The spore or particle size limits the drift to open joints with a minimum opening width and karst cavities. Porous sediments, rocks or materials with a smaller pore size hold back the spores or polystyrene beads.

Activation-analytical tracer method: For feeding into the sinkhole, a non-radioactive substance is added to the infiltrating water and only the water samples taken are placed under neutron bombardment to activate the element in the reactor. Bromine as ammonium bromide (NH₄Br), lanthanum or indium are possible tracer substances. In the case of indium, a tracer amount in the range of several 100 g can be expected (ZÖTL, 1974). The advantages of this method are the use of non-toxic, non-radioactive substances, which only have to be used in relatively small amounts. As a result, no special safety measures are required during the test procedure. As with the other tracer methods, the evaluation is carried out using a concentration/time diagram. The interstitial velocity of the groundwater can thus be determined between the location of the tracer input and the location of the tracer measurement.

Single-borehole dilution method with radioactive tracers: The decrease in the concentration of a previously added tracer is measured in a borehole region blocked by packers. A dilution rate can be derived from the decrease in concentration over time in the packer interval. The dilution rate is empirically related to the prevailing filter velocity. Corresponding to the borehole construction (filter pipe type, slot width, etc.), the measurement results must be corrected. If radioactive material is used as the tracer, the horizontal groundwater flow direction can be determined by determining the radioactive radiation by an angle-dependent detection after the tracer has flowed into the aquifer. However, radioactive tracers are problematic in drinking water supplies.

Single-borehole dilution method with uranine as a tracer measurement method: The Institute of Hydraulic Engineering at the University of Stuttgart (MARSCHALL 1993) has developed a light guide fluorometer for in-situ concentration measurement. Thereby, fluorescent dyes such as uranine can also be used. The detection limit for these substances is much lower (10⁻⁵-10⁻⁶ mg/l) than for conductivity measurements with NaCl tracers (0.1-1 mg/l). A horizontal filter velocity of 10⁻⁷ m/s can be measured thereby.

The use of a dye as a tracer is also described. The tracer substance is put into an upstream gauging station (measuring point) or sinkhole (sinkage section) as a dye for analysing currents and flow direction. The Danube Sinkhole was already cleared up in 1877 with fluorescein as the tracer molecule.

The groundwater velocity is significantly influenced by the interconnected rock properties, such as rock mass permeability (water permeability in the natural rock structure, rock crevices and flow-effective cavity volume). However, at the beginning of the location selection, data on these variables are neither available comprehensively nor can they be collected for all locations to be considered with sufficient evidence.

Disadvantages are the low horizontal depth of penetration (range) into the rock mass formation or porous materials. The storage coefficient can only be estimated. Statements about the aquifer model, aquifer margins and anisotropy are only possible to a limited extent with special evaluation methods. The accuracy of the measurement results primarily depends on the quality of the data acquisition, but not on the tracers used per se. Precise pressure and volume measurements are crucial, particularly in the case of low permeability.

Such measurements in stone cracks, rocks, geological formations, porous materials or in boreholes to measure parameters such as pressure, temperature on site are difficult or impossible due to the inaccessibility and may only be suitable for very small measuring probes. Miniaturized electrical or optical sensors and measuring devices that can measure in such locations and under such conditions can only be manufactured with a great deal of effort and expense and are difficult to introduce into the rock and to connect to external measuring electronics.

A conventional determination with measuring probes, sensors and detection electronics is usually ruled out because, on the one hand, they have to be supplied with energy and, on the other hand, the data has to be read out. Both would require a supply with power and signal lines, which would require separate drilling, which would not only require enormous technical and financial expenditure, but would also falsify the measurement parameters and the flow conditions (e.g. of water in these rock strata). The use of prior art tracers only leads to a passive measurement of parameters such as flow velocity, dilution or permeability.

A quantification of parameters such as pressure, temperature or other physical and/or chemical parameters as they pass through the rock and/or rock strata or porous materials, or which physical, chemical and biochemical conditions they were exposed to on their way, are not recorded.

### SUMMARY OF THE INVENTION

Based on this, it is the object of the invention to provide a novel method for quantifying a porous medium, by which the above-mentioned disadvantages of the conventional methods can be avoided.

The invention relates to a method for quantifying porous media and to the analytical particles specially designed therefor and to the use thereof, for example in order to determine the water permeability of rocks as a prerequisite for the development of criteria for ground water movement or the material characterization of porous materials or rock layers or for monitoring chemical, biological and/or biotechnological reactors, water tanks, water reservoirs and water line systems or in medical in-vivo methods. A fluid is also provided.

### DETAILED DESCRIPTION OF THE INVENTION

The object was achieved by a method for quantifying a porous medium with at least one particle or a mixture of particles, wherein the particles have a reference function and at least one reporting function for recording physical, chemical or biochemical parameters of the porous medium, said method comprising the following steps:
introducing the particle and/or the particle mixture into a fluid,
letting the fluid with the particle and/or the particle mixture flow through and/or permeate the porous medium, wherein the at least one reporting function of the particles changes when a threshold value of the parameter to be recorded is exceeded or fallen below, while the reference function of the particles remains unchanged, and
after exiting the porous medium, at least one subsequent analysis of the particle and/or the particle mixture for the physically, chemically or biochemically changed reporting function and the reference function of the particles, wherein the reference function serves to recognize the particles.

The particle according to the invention is on the sub-millimeter scale, the micrometer scale or the nanometer scale and comprises at least one reference function and at least one reporting function for recording physical, chemical or biochemical parameters of the porous medium.

The particles preferably have a diameter of 100 μm to 0.5 nm, particularly preferably a diameter of 10 μm to 5 nm, or very particularly preferably a diameter of 5 μm to 50 nm.

The base body of the particles preferably consists of silver, gold, copper or other metals, silicon dioxide, polystyrene, olefins, wax or a mixture thereof.

The at least one reporting function is preferably contained in the particles and/or on the particle surface.

According to the present invention, the reporting function comprises at least one fluorescence marker, a luminescence marker, a marker for the plasmonic property, a pH value indicator, a temperature indicator, a radiation indicator alone or in combination with one another.

The change in the at least one reporting function is preferably irreversible.

The change in the at least one reporting function preferably increases continuously with the dose of radiation (radiation exposure) or the oxidative stress experienced.

The reference function of the particles is preferably in the form of a geometric and/or haptic detection site, or in the form of a fluorescence marker, a luminescence marker or a marker for the plasmonic property.

The particles preferably further have an additional function that allows determining the residence time of the particle in the porous medium on the basis of a time-dependent disintegration or a time-dependent change in a property.

The particles preferably further have a magnetic additional function.

The particles preferably have a further reporting function that changes when a threshold value of a second parameter to be recorded, which is different from the first parameter, is exceeded or fallen below.

The porous medium is preferably a liquid- or gas-filled space.

It is further preferred for that the porous medium to comprise rocks, rock strata and/or a porous material or layers made of this porous material.

The analysis of the particle and/or the particle mixture is preferably carried out by optical spectroscopy, IR spectroscopy, plasmonic resonance, microscopy, dosimetry, nuclear magnetic resonance, electron spin resonance, ENDOR, fluorescence spectroscopy, single-molecule fluorescence spectroscopy, atomic fluorescence spectroscopy, luminescence spectroscopy, photoluminescence spectroscopy, chromatography, gas chromatography, liquid chromatography and/or high-performance liquid chromatography (HPLC), or by means of a follow-up reaction which facilitates the detection of the change in the reporting function.

The present invention further relates to the use of the method according to the invention for quantifying rocks, rock strata and/or porous materials or layers of this porous material, in the geological analysis of rocks, rock strata and/or porous materials or layers of this porous material, in hydrology, water exploration, deposit exploration, deposit monitoring, fracking, geothermal energy, leakage monitoring, monitoring of chemical, biological and/or biotechnological reactors, of water tanks, water reservoirs and water supply systems, or in medical in-vivo methods.

The present invention also relates to a fluid with at least one particle according to the invention or a mixture of the particles according to the invention for use in an in-vivo medical method.

As described above, the particle has a reference function and at least one reporting function for recording physical, chemical or biochemical parameters. The reference function represents a reporting function unchanged by the physical, chemical or biochemical parameters and serves to detect the particle(s) after it/they has/have exited the porous medium.

The reporting function changes as soon as the conditions that the particle experiences as it passes through the medium are such that a condition (a physical quantity such as temperature, pressure, light, radiation of a specific intensity or wavelength etc. or a chemical quantity such as pH value, ionic strength, concentration of a specific anion or cation, a specific solubility product, the concentration of a specific chemical species, for example a specific molecule, ion or radical) exceeds a specific threshold value (or limit or critical value) or falls below it.

According to the present invention, the final analysis of the particle and/or the particle mixture for the reporting function takes place after it has passed through the porous medium, i.e. after the flow and/or permeation through the porous medium (hereinafter also generally referred to as “passage”), and has exited the porous medium. Here, “exit” does not mean that the whole of the particle must be spatially separated from the medium. Rather, “after exiting the porous medium” is to be understood to mean that the analysis of the particle and/or the particle mixture takes place at a location that is different from that at which the physical, chemical or biochemical parameters of the medium to be examined are to be recorded.

As a result, according to the invention, information about the parameters is acquired by the particles in situ (e.g. in rock, in the geological formation or in a chemical reactor) and analyzed ex situ after passing through these locations.

The particle is therefore an analysis particle (tracer), preferably micro and/or nanoparticles. These have a reference function (also referred to as an identification function) in the form of a mark, which is independent of the physical, chemical, and biochemical parameters experienced and which enables the detection of the particles after their passage or permeation through the medium. The particles also have at least one reporting function (i.e. another marking function, also referred to as a reporting function or story-telling function), which changes, preferably irreversibly, depending on the physical, chemical and biochemical parameters experienced. The reporting function comprises a detection and/or quantitative and/or qualitative recording of the physical or chemical and biochemical parameters experienced during the passage or permeation through the medium, preferably by means of a quantitative and/or qualitatively analyzable signal of the substances and/or liquids used for the reporting function.

With the aid of the method according to the invention, areas and locations can be analyzed that are otherwise difficult to access or inaccessible and are not in visual contact with the observer, preferably inside a geological formation, i.e. not on the surface of the earth, or inside a reactor or a human or animal body.

The reporting function can either be contained inside the particle or provided on its surface.

According to the invention, any measures can be taken to implement the reporting function. However, the reporting function preferably comprises at least one fluorescence marker, a luminescence marker, a marker for the plasmonic property, a pH value indicator, a temperature indicator or a radiation indicator. These can be present individually or in combination. The reporting function can be shielded to the outside with a semipermeable or permeable membrane as a separating layer or changeable layer and/or to the subsequent or preceding reference function.

The reporting function can preferably comprise chemical markers or biomarkers that detect the presence of a specific chemical or biological species.

Alternatively or in combination, a quantitative and/or qualitative change in the particle itself is used as a reporting function. To this end, the reporting function or the particle itself undergoes a change due to the treatment of the surrounding physical, chemical or biochemical parameters. This change occurs upon passage through the porous medium. The particles or particle mixtures are not present in the natural ecosystem, but are introduced as part of the method according to the invention.

As described above, the particles are introduced into a fluid. In general, the fluid passed through the porous medium can be not only a liquid, but also a gas, a gel or a liquid-gas mixture, an emulsion, a mist (liquid droplets are carried with a gas) or an aerosol. Consequently, the fluid may be water, oil, an oil-water mixture, a generated or natural gas stream, a liquid-gas mixture, vapors, and also mist.

The particles, in particular nanoparticles, with the reference and reporting function can be in any component of such mixed fluids. They can be in one component or in more than one component, for example both in the liquid droplets and in the gas phase of a mist or only in one of the two.

The fluid may also contain additives and/or detergents. The gas stream preferably consists of air, industrial gases or noble gases or mixtures thereof.

The analysis of the particle and/or the particle mixture is preferably carried out by optical spectroscopy, IR spectroscopy, plasmonic resonance, microscopy, dosimetry, nuclear magnetic resonance, electron spin resonance, ENDOR, fluorescence spectroscopy, single-molecule fluorescence spectroscopy, atomic fluorescence spectroscopy, luminescence spectroscopy, photoluminescence spectroscopy chromatography, gas chromatography, liquid chromatography and/or high-performance liquid chromatography (HPLC).

A variant of the method according to the invention removes the particles or the fluid with the particles and carries out a follow-up reaction that facilitates the detection of the change in the reporting function. One way of detection is, for example, using test strips, similar to what is common in medicine and pharmacy, on which the corresponding detection reagent is located. This may e.g. change color when the reporting function of the particles has changed upon passage through the porous medium.

The method according to the invention is used for the quantification of rocks, rock strata and/or porous materials or layers of this porous material, in the geological analysis of rocks, rock strata and/or porous materials or layers of this porous material, in hydrology, water exploration, deposit exploration, deposit monitoring, fracking, geothermal energy, leakage monitoring, monitoring of chemical, biological and/or biotechnological reactors, of water tanks, water reservoirs and water supply systems, or in medical in-vivo methods.

In particular, the method according to the invention is suitable for hydrological and/or geological analysis of rocks, rock strata and/or porous materials or layers of this porous material.

According to the invention, the following parameters are recorded by direct or indirect detection of physical, chemical or biochemical parameters on the individual particles according to the invention by the reporting function:
Physical conditions, for example pressure, temperature, radiation, or also chemical conditions, for example pH, as a measure of the acidic or alkaline character of an aqueous solution, or the ion concentrations of a chemical compound or an element.
Concentration, type and/or occurrence of gases such as oxygen, nitrogen, gaseous compounds of elements of the periodic table (periodic table of elements) or gaseous hydrocarbons.
Concentration, type and/or the occurrence of elements of the chemical periodic table, their salts, ions or their covalent compounds among each other, for example alkali metals, non-metals, metals, semimetals, noble gases, lanthanides or actinides.
Concentration, type and/or the occurrence of organic macromolecules or organic-inorganic macromolecules, their salts or ionic compounds, for example fats or oils.
Concentration, type and/or the occurrence of biomolecules, for example humic acids, proteins, nucleic acids or ribonucleic acids.

The physical, chemical or biochemical parameters on the individual particle are recorded on-site on the inside when the porous medium is flown through and/or permeated.

Permeation or diffusion is understood to mean the process of penetration, passage or traversing, in which a substance (the particle according to the invention) as a permeate penetrates, passes through or traverses a solid as a porous medium. The driving force is a concentration or pressure gradient of the permeate or the Brownian molecular motion.

Flow means the process of traversing voids and/or interstices between solids.

Due to the influence of the physical, chemical or biochemical parameters, the particle receives at least one signal which the particle picks up and incorporates at least through an analytical reporting function (i.e. external marking function and/or internal cavity filling) and/or through a change in shape of the particle itself, so-called memory effect. For this purpose, the particles, unmixed or in mixtures of different particles, are introduced into the medium by means of a liquid or gas stream (e.g. in rock cracks, rocks, geological formations, channels or pores) and changed in the particle volume or on their surfaces or the particle layers by the physical, chemical and biochemical environmental conditions experienced.

According to the invention, particles are used on the millimeter scale (5-1 mm), the sub-millimeter scale (999 μm to 100 μm), the micrometer scale (100 μm to 1 μm) or the nanometer scale (999 nm to 0.4 nm).

Water, oil or oil-water mixtures, with or without the addition of additives and detergents, can be used e.g. as the flowing and/or permeating liquid that serves as carrier material or flow and/or permeate liquid of the particles according to the invention. A generated or natural gas stream, for example from air, industrial gases or noble gases, can also be used as the carrier material of the particles according to the invention. Liquid-gas mixtures or vapors, mists (gas phase plus condensate) are also suitable as the fluid.

The diameters of the particles used are preferably in the micrometer to nanometer range, i.e. between 0.5 nm and 100 μm, particularly preferably between 10 μm and 5 nm. Particles between 50 nm and 5 μm are preferred. Depending on the geological or hydrological analytical question, the particles are used unmixed or in mixtures.

Particles that can be easily and inexpensively produced in large quantities and with a narrow size distribution (dispersity) consist of oxides or of polymers or of wax. For example, they can be made from silicon oxide or polystyrene or from olefins. In the particles or on their surface, molecules or smaller nanoparticles can be incorporated, accumulated, detached or attached with the desired reference or reporting properties.

Another very relevant class of materials are particles made of metal, such as silver or gold. By adsorption of, for example, thiols or dithiols, but also of other molecules that change the plasmonic property, the plasmonic properties of metallic nanoparticles can be changed significantly and thus be detected, or the signal can be used as a reporting function. Moreover, changes can be detected by chemical reactions of thiols or dithiols chemisorbed on the surface of the metal nanoparticles by change in the plasmonic properties—(i) intensity of the plasmonic absorption, (ii) width of the corresponding absorption band and (iii) position of the absorption maximum. The metallic nanoparticles combine reporting and reference properties in a very advantageous way: the “naked” nanoparticles also show the plasmonic resonance. However, it is changed by the adsorbates and chemisorbates: the shifted resonance develops and the resonance decreases with the original resonance spectrum.

Metallic nanoparticles, for example made of silver, gold or copper, are therefore preferably used as the material for the particles. Adsorbates, chemisorbates and the interaction with the surrounding medium change their plasma resonance, which can be measured spectroscopically. These particles are easy to manufacture and are easily commercially available.

As the material for the particles, polymer particles are also preferably used, in particular the very easy-to-produce and commercially available polystyrene latex spheres or silicon oxide particles (silica spheres), which are also easily produced with a narrow size distribution and in a spherical form and are also commercially available.

The particles can have different shapes and forms. For many applications, preference is given to spherical particles (for example polystyrene latex spheres or silica spheres), or to round, elongated, rounded or rod-shaped particles, for example in the form of micelles, designed as hollow structures, filled structures or core-shell particles. Also possible are platelet-shaped structures, e.g. mineral discs made of mica or laponite, which can be functionally coated, and—in particular in the field of polymers and biopolymers—also irregularly shaped structures. In the case of polymer and biopolymer particles, their (i) folding and (ii) conformation as well as (iii) their degree of swelling can also be used for the detection. Many polymers tend to coagulate in solution, which loosens under specific conditions such as pH or ion concentration. All three parameters mentioned depend strongly on the ambient conditions. However, these changes are mostly reversible. When increasing the degree of swelling or loosening the coagulation, embedded molecules are either released or changed by contact with the fluid (e.g. the surrounding water) in such a way that they are irreversibly released and/or changed, and this change can be detected as a reporting property, for example, with optical spectroscopy, infrared spectroscopy, ultraviolet spectroscopy or fluorescence spectroscopy.

In addition to (1.) classic core-shell particles, in which the particle is surrounded by a shell in the form of a coating, for example particles of silica, covering made of polystyrene or another polymer or covering of a particle with wax, paraffin or a layer of fat that melts at a specific temperature, it is also possible to use (2.) particles that consist of a core and a molecular layer enveloping the core—preferably without gaps. An example of this is the covering of a metallic micro- or nanoparticle, preferably made of coin metals such as copper, silver, gold, but also of nanoparticles made of metal alloys, with a layer of thiols, dithiols and thiol derivatives. However, particles of oxides of metals and metal alloys, of silica, aluminum oxide, titanium dioxide and also particles of polymers are also conceivable here, which are surrounded by a monomolecular layer. (3.) Moreover, it is also possible to use different types of hollow particles in which the inner cavity is completely or partially filled with a) at least one gas, b) the surrounding fluid, for example water, or c) a fluid other than the surrounding fluid, e.g. oil, fat, silicone oil and/or a fatty acid. The aforementioned substances or chemical substances and/or particles contained therein preferably have a marking function as a reporting and/or reference function. Or d) a porous substance with gas-filled cavities or e) a porous substance with liquid-filled cavities.

If the shell is rigid and fragile, but the core is compressible, the shell breaks under pressure and the interior is exposed to the surrounding fluid (e.g. water). If an irreversible chemical reaction now occurs between a water-sensitive component inside the particle and the water, this component will change at least one physical or chemical property, which can then be used as a reporting property in the sense of the present invention. In this way, it can be demonstrated that the pressure required to collapse the particle on its way (e.g. through the rock) was reached. The threshold pressure, i.e. the minimum pressure that must be reached so that the particle breaks or collapses under external pressure, can be set very easily, and it is possible to produce customized particles with different threshold pressure. The pressure at least to be reached until the particle collapses increases for the given substance for the shell and given filling with increasing shell thickness and decreasing particle diameter.

According to the invention, at least one analytical particle (tracer) with a reference and reporting function is used. A combination of several analytical particles with or without a porous separation layer is also possible. For example, particle mixtures consisting of a filled particle and a particle with a central cavity and a surrounding shell, the marking function with the reporting function and/or the reference function in each case (i) being applied to the shell, (ii) integrated into the shell or (iii) being located within the cavity.

However, a combination of several identical or different marking functions (as a reporting and/or reference function) with or without permeable separating layers and with or without a permeable outer shell is also possible, wherein also the marking function or an analytical tracer itself can represent the outer shell of the particle. The cavity can optionally contain a gel-like, pasty or gaseous filling (see above). This filling can itself serve as an analytical medium or change the physical properties of the particle, such as weight or buoyancy properties, such as static buoyancy, for example by collapsing the shell after reaching (exceeding or falling below) a specific pressure, pH value, temperature etc.

The three aforementioned material systems are, however, only the starting particles for the analytical particles with the marking function, in the manner of a reporting function and reference function, to be produced according to the invention. The implementation of the reporting and reference function for the respective particles takes place by applying an adsorbate or chemisorbate shell to the surface of the particles and/or by introducing the reporting and reference functions—for example by introducing suitable chemical molecules as a marking function—into the inside of the particles, for example during the particle production process or by subsequent diffusion and/or filling of a hollow volume of the particle. The particle can also be produced in two half-shells, which can be filled with the respective marking function, and then two half-shells can be connected in a final connection.

Two specific exemplary embodiments for the particles with the reference function and the at least one reporting function will be described in more detail below.

Core-Shell Version:

The particle is surrounded by a shell made of a different material or of the same material in a different structure. For example, the core may include the reference function, the shell the reporting function, or vice versa. The variant in which the reporting function is in the core and reacts to contact with water is particularly interesting. The shell protects the core from water. As long as the shell exists and is diffusion-tight for water, the reporting function in the water-sensitive interior (core) does not change. If the core is surrounded by a water-diffusion-tight shell, which becomes permeable to water when a specific threshold condition is reached (reaching a specific temperature, exceeding or falling below a specific pH value, carrying out a specific chemical or biochemical reaction), this can be detected by the change in the reporting function in the core of the particle through contact with the water. This can preferably be done in four different ways:

First, the shell becomes porous when the threshold condition is reached.

Second, the shell loses its diffusion tightness against water when the threshold condition is reached.

Third, the shell dissolves when the threshold condition is reached.

Fourth: The shell detaches from the core when the threshold condition is reached.

An example is the melting of the shell when a specific temperature is reached or the dissolution of the shell when a specific pH value is reached.

Particle-in-Particle Variant:

In addition to the core-shell version, the present invention can be implemented by a particle-in-particle variant. Smaller particles are introduced into the larger particles, for example metal nanoparticles in polymer particles, for example by a precipitation reaction. The shape of the particles may be round or spherical. By the shape of the particles, the properties of the particle for dynamic buoyancy in the fluid or the flow or permeation of the porous medium can be influenced and adapted.

According to the invention, the particle can have a further additional function in addition to the reference and reporting function. For these trifunctional particles, nanoparticles with the diameters mentioned above are also preferred. This additional function may be a timer function that allows determining the time of the particle from the injection to the detection after leaving the porous medium on the basis of a time-dependent disintegration or a time-dependent change in a property. Radioactive decay is just one example. Chemical decomposition, the conversion of one isomer into another, more stable isomer or an oxidation with loss of the fluorescent property are further examples.

This additional function can further be a magnetic function. The use of trifunctional nanoparticles, the third function of which is their magnetic properties, preferably their ferromagnetism. This can be realized, for example, in that the particles also contain magnetic inclusions in addition to their reporting function and their reference function. Later on, this facilitates the concentration and removal of the particles from the fluid using magnets and magnetic fields. For example, after leaving the porous medium, the fluid can flow through a grid or mesh that has magnetic properties and attracts and holds the particles.

Metallic nanoparticles, preferably made of gold or silver, exhibit a plasmonic resonance, which can be detected with optical spectroscopy or IR spectroscopy. This resonance shifts when specific chemical molecules, preferably thiols, are adsorbed. The presence of the metallic nanoparticles can always be detected spectroscopically by the existence of the plasmonic resonance, as a reference function.

The question of whether the particles encountered thiols on their way through the porous medium can be determined from the spectral position of the resonance after passing through the medium: When the thiols are adsorbed, the spectral position of the plasmonic resonance shifts. The proportion of particles that encountered the thiols can also be determined in this way. The plasmonic spectrum can be broken down into a portion that is not shifted and a portion that is shifted.

The reversible or partially reversible implementation of the reporting function will be described below, with a (largely) irreversible change in the reporting function being preferred due to the conditions experienced. If you have a reporting function that has changed due to a specific condition experienced and that, when this condition no longer exists, slowly changes back to its original state, you can conclude how long ago the experience occurred and thus indirectly where, for example in a layer of rock, the conditions mentioned prevailed.

A special embodiment is the use of a bifunctional molecule as a particle with a reporting unit and reference unit, hereinafter also referred to as a “two-in-one” solution. Here, the reporting and reference unit are not separate units, but are united in one unit.

Example: Metallic nanoparticles, for example made of gold or silver, exhibit a plasmonic resonance that can be detected with optical spectroscopy or IR spectroscopy. When specific molecules (e.g. thiols) are adsorbed, this resonance shifts. The presence of the metallic nanoparticles can always be detected spectroscopically by the existence of the plasmonic resonance (reference function). The question of whether the particles encountered thiols on their way through the porous medium can be determined from the spectral position of the resonance after passing through the medium: When the thiols are adsorbed, the spectral position of the plasmonic resonance shifts. The proportion of particles that encountered the thiols can also be determined in this way: The plasmonic spectrum can be broken down into a portion that is not shifted and a portion that is shifted. The reversible or partially reversible implementation of the reporting function: Basically, the entire and/or partially irreversible change in the reporting function by the conditions experienced is thought of. If you have a reporting function that has changed due to a specific condition experienced and that, when this condition no longer exists, slowly changes back to its original state, you can conclude how long ago the experience was and therefore also indirectly where (for example in a rock strata) the conditions mentioned prevailed.

Deviating from the above point, it would be possible in exceptional cases that the particles or molecules are naturally already present in the system and are not introduced as part of the method according to the invention. A practical example of pH-dependent particles are particles that contain molecules or molecular groups that split above a specific pH value (i.e. when it is exceeded) or split off a specific molecular group. Another example of pH-dependent particles are particles that contain molecules or molecular groups as a reporting function that split below a specific pH value (i.e. when it is fallen below) or split off a specific molecular group. These reactions are practically completely irreversible, since if the critical pH value is exceeded or fallen below again, the group required for the reverse reaction is no longer available.

Particles for the detection of a temperature overshoot are particles with a temperature-independent core that is resistant to higher temperatures and a shell that melts and detaches from the core when a specific temperature is exceeded (threshold condition). Here, the particle has several markings with a characteristic geometric shape, for example a cone, which change at different softening temperatures in the medium flowing through. Whenever a specific temperature is reached, a specific marking becomes soft and changes its shape. It is clear from this what temperature has been reached in the medium and what has not yet been reached. The core may be a metal particle, an oxidic particle, for example made of silicon dioxide, aluminum oxide or titanium dioxide, or a polymer particle, for example polystyrene. The shell may consist of any substance that is not water-soluble in the solid state and that melts at the desired temperature. These can be waxes, paraffin, long-chain olefins or alkanes or also fatty acids, aldehydes or esters. If the melting temperature of the respective substance from which the shell is formed is reached, the shell will be detached in the flow or the molecules of the shell dissolve in the water or in the polar fluid.

According to the invention, it is also possible to combine different particles that are sensitive to temperature overshoots of different temperatures. For example, the use of 10 different particles whose shells melt at 10° C. (degrees Celsius), 20° C., 30° C., 40° C., 50° C., 60° C., 70° C., 80° C., 90° C. and 100° C. and who each use a different molecule as a reference. In this way, detailed conclusions as to which temperatures have been exceeded and which have not, and what percentage of the particles have experienced a temperature overshoot on their way through the porous medium and thus, for example, have lost their shell in the embodiment mentioned, can be drawn in 10-degree steps.

A further embodiment is that 10 different particles with 10 different melting points of the shell are introduced simultaneously or sequentially and the flow and/or permeation are observed or analyzed chromatographically as a function of time. In this case, the reference property is the same for all 10 particle types. In an analogous manner, of course, the examinations of different properties (such as exceeding and/or falling below the pH value, exceeding the temperature and exposure to oxygen) can be analyzed simultaneously or sequentially. Another embodiment of particles for detecting a temperature overshoot in non-aqueous weakly polar or apolar media are particles with a temperature-independent core that is resistant to higher temperatures and a shell that melts and detaches from the core when a specific temperature is exceeded (threshold condition). The core may be a metal particle, an oxidic particle (for example made of silicon dioxide, aluminum oxide or titanium dioxide) or a polymer particle (for example polystyrene), the shell may consist of an arbitrary substance that is not oil-soluble in the solid state and melts at the desired temperature. These can be polar molecules such as polyhydric alcohols and/or long-chain or short-chain carboxylic acids. If the melting temperature of the respective substance from which the shell is formed is reached, the shell will be detached in the flow or the molecules of the shell dissolve in the oil or in the apolar fluid.

A practical example of pressure-dependent nanoparticles are so-called “hollow spheres”, hollow particles in which there is gas and no liquid and which collapse under a specific hydrostatic pressure. Different collapse pressures can be set depending on the diameter and wall thickness. The particle volume can be partially or completely filled with a solid, gel-like, pasty or gaseous cavity filling.

The type of change in the reporting function will be explained in more detail below. This can be done irreversibly (hereinafter referred to as “threshold value variant”) or similar to a dosimeter.

The threshold variant: If, for example, a specific temperature is reached, the reporting function of a specific particle type changes. If a further temperature is reached, the reporting function of another particle type changes. It can therefore be demonstrated whether a specific parameter value has been reached, exceeded or fallen below.

The dosimeter variant: The extent of the change depends on the strength and the time of exposure. The change in the reporting function due to radioactive radiation or UV light is mentioned as an example. The extent of the change is then dependent on the dose, i.e. proportional to the radiation intensity and the exposure time—which corresponds to the invention of the possibly smallest dosimeter in the world, the (micro- or nano-) particle dosimeter. Analogously, a chemical dosimeter variant is also conceivable. The chemical dose is then the concentration of the chemical or biochemical species to be detected times the time of exposure. In the simplest case, this can be the dose of the action of oxygen, i.e. the oxidative dose caused by oxygen. As an extension of this method, there can of course also be different oxidative species, which can each cause an oxidation of the particle's reporting function, and the total oxidative dose can be determined. In apolar media, such as oil, emulsified water droplets or other droplets of a polar liquid can be used instead of the particles, as can be micelles. The droplets or micelles can then contain molecules, molecular groups or particles or nanoparticles with reference properties and with reporting properties in exactly the same way.

In the chromatographic analysis of the data, the particles are not injected continuously, but at a specific point in time, and come out again e.g. after different dwell times in different paths through the fractured rock or the porous medium in order to be examined at the exit point as a function of time. Different dwell times correspond to different paths through the rock or the porous medium (possibly with dwell time after “trapping” in a pore). The “experience report” of the particles after exiting the porous medium as a function of the dwell time provides information about how high the percentage of the particles with exposure to the requested conditions (e.g. temperature above 80° C.) was as a function of the dwell time.

The particles can selectively also be provided with a respective characteristic reference function (recognition function) in the form of a marking, for example a marking with a geometric and/or haptic shape, similar to a fingerprint. Another example is marking with a fluorescent dye that does not occur in nature and whose absorption or fluorescence spectrum represents a fingerprint for the presence of this molecule. If the particle contains this fluorescent dye inside or bound on its surface, it can be identified without any doubt. A preferred identification is given by the simultaneous use of two fluorescent markers in a particle for recognition (double recognition function).

The particle can thus be recognized as a particle of a specific injection batch at any time. If you bring in differently marked particles at different times or in different places, you can—if necessary even after years—recognize where these particles come from. If these particles exit again, they carry information about the environmental conditions experienced during their passage or time in the rock due to this change. Such particles can be introduced billions of times. By combining particles that are changed by different environmental conditions (threshold temperature, pressure, etc.), different environmental conditions experienced can be queried at the same time. This makes it possible to analyze that, for example, 70% of the particles have encountered temperatures above 250° C. and 20% of the particles have encountered temperatures above 300° C. and 44% of the particles have encountered pH values below 4.5.

However, in addition to the reference function, it is also possible to equip the particle with more than one reporting function. An example is a nanoparticle that loses its shell and thus also the fluorescent dyes of fluorescence wavelength 1 as reporting function 1 when a specific temperature is exceeded (the melting point of its shell with which it is encased). At the same time, the core of the particle encased with the meltable shell (e-g. a wax shell) contains another fluorescent dye that fluoresces at wavelength 2 and is chemically stable under the conditions in the porous medium as a reference function. In addition, the particle core also contains a further fluorescent dye, which fluoresces at wavelength 3, irreversibly changes its fluorescence when the pH value is fallen below or exceeded as a threshold value and then no longer or not that strongly fluoresces at this wavelength 3, or fluoresces at a new wavelength 4.

According to a preferred embodiment of the present invention, use if made of particles whose reporting function changes when the threshold value is exceeded or fallen below, such a threshold value being a sharp value or a narrow range of values, for example a melting temperature of the wax shell stained with fluorescent dye of +/−10° C., preferably +/−5° C., particularly preferably +/−3° C.

Particularly preferred embodiments of the method according to the invention use particles that are characterized by the following features:

1. Particles with a particle core and an inner and an outer shell,
wherein (1.) the particle core contains the reference function (for example fluorescent or when irradiated with electromagnetic radiation, e.g. light, shows defined electromagnetic resonances, e.g. optical resonances, or that the particle core contains components showing this, for example fluorescent molecules or quantum dots or metallic nanoparticles that show plasmonic resonances),
wherein (2.) at least one component is incorporated in the inner shell, which includes the reporting function and is water-soluble or oil-soluble, and
wherein (3.) this inner shell is surrounded by an outer shell that is diffusion-tight with respect to the surrounding fluid, so that the component (having the reporting function) incorporated in the inner shell can neither be detached from the fluid nor chemically changed or modified, swollen, etc. in some other way, wherein the outer shell is characterized in that, when the threshold value of the parameter to be detected is exceeded or fallen below, it is either open to diffusion for the fluid or for the component enclosed in the inner shell or for both or by the outer shell dissolving when the specified threshold value is fallen below of or exceeded, thus releasing the components incorporated in the inner shell and having the reporting function.

2. Particles according to 1., characterized in that the particle core is a micro- or nanoparticle made of metal (preferably silver, gold, lead, copper, iron, cobalt, a metal oxide or a ferromagnetic material such as iron oxide or cobalt oxide) or silicon dioxide or carbon or polystyrene.

3. Particles according to 1. or 2., characterized in that the outer shell is a meltable shell that melts or softens at a specific temperature or in a specific temperature range or dissolves in the surrounding fluid. Alternatively, use can also be made of a shell that dissolves, softens, swells or is open to diffusion when a specific pH value or a specific ion concentration is exceeded or fall below.

4. Particles according to 1. to 3., characterized in that this outer shell consists of paraffin or of metal or of an organic or inorganic polymer or oligomer meltable or softenable or swellable in a specific temperature range.

5. Particles according to 1. or 2., characterized in that the outer shell is a shell made of an oil-soluble substance that dissolves upon contact with oil.

6. Particles according to 1. or 2., characterized in that the outer shell is a shell made of a polymer brush that is grafted onto the inner shell, or that the outer shell is a coordination polymer.

7. Particles according to 1., 2, or 6., characterized in that the outer shell is a shell made of a polyelectrolyte. In a special embodiment, it can also be grafted onto the inner shell. The outer shell can also be a crosslinked or photo-crosslinked polymer.

8. Particles according to 1. to 7., characterized in that fluorescent dyes or quantum dots or magnetic nanoparticles are incorporated in the particle core.

9. Particles according to 8., characterized in that at least one of the fluorescent dyes used is rhodamine or rhodamine derivatives or GFP (green fluorescent protein) or ruthenium-bipyridine-based complexes and compounds (e.g. Rubpy).

10. Particles according to 1. or 7., characterized in that the outer shell is a polymer or polyelectrolyte that is open to diffusion above a specific pH value or a specific temperature.

11. Particles according to 1., characterized in that the outer shell is a semi-permeable membrane.

12. Particles according to 1 to 11, characterized in that the particle has more than two shells in addition to the particle core.

According to the invention, the reference function in the form of the marking is preferably embedded in the particle core and is not dissolved out by the fluid. The at least one component having the reporting function, which is incorporated in the inner shell, can be dissolved out by the fluid upon contact with it. As a result, when a fluid in which it is soluble comes into contact with the inner shell, it can be “removed” again.

The method for quantifying rocks, rock strata and/or porous materials or layers made of this porous material, for the geological analysis of rocks, rock strata and/or porous materials or layers made of this porous material will be described in more detail below. Rock is a solid, naturally occurring, usually microscopic heterogeneous association of minerals, fragments of rock, glasses or residues of organisms. The mixing ratio of these components to one another is largely constant, so that a rock, despite its detailed composition, looks uniform when viewed with the naked eye. A detectable change in properties is caused by specific physical, chemical or biochemical environmental conditions of the material to be examined. If the particle encounters such environmental conditions on its way through the porous medium or rock (example: a temperature of at least 80° C.), the particle will be changed irreversibly, which is detectable by a change in properties after it has exited the porous medium or rock. This change in properties of the particle is now detected after flowing through and/or permeation of the rocks, rock strata and/or porous materials or layers made of this porous material to be examined. This is done, for example, in the following way. At the exit point of the fluid, either samples are taken for this purpose and the fluid with the particles is examined, or samples are taken for this purpose and the particles are enriched—for example by centrifugation or ultracentrifugation or by sedimentation—and then examined. Separately or in combination with the sampling, the properties can be examined in real time in the flowing liquid, for example through glass windows in the flow cell of a spectrometer through which the fluid flows. The change in properties of the particles can affect very different properties: This can be, in particular, the absorption of electromagnetic radiation, such as light, ultraviolet radiation or infrared radiation or microwave radiation. However, it can also be a change in the magnetic properties, for example of ferromagnetic or superparamagnetic particles and nanoparticles, which can take place, for example, through oxidative change due to the action of oxygen. However, it can also be a change in the dielectric properties, which can likewise take place, for example, through an oxidative change due to the action of oxygen, but also through another chemical change in the particle or its surface or of the molecule in question.

Depending on the type of particles used, changes in particle properties can also be detected by NMR and ESR (nuclear magnetic resonance or electron spin resonance). The changes in properties can also be detected by means of ENDOR (electron nuclear double resonance) using magnetic resonance.

However, there can also be a change in the fluorescence properties, which is possible in different ways: change in the fluorescence intensity or change in the fluorescence wavelength or occurrence of new fluorescence or occurrence of the fluorescence at a different excitation wavelength than before. The latter is particularly preferred for the detection described below. At a certain excitation wavelength, fluorescence occurs after flow through the porous medium, which did not exist before flow through the porous medium, or only with a lower intensity. Alternatively, at a specific excitation wavelength, fluorescence occurs before flow through the porous medium, which no longer exists after flow through the porous medium, or only with a lower intensity.

After the particles and/or molecules have flown through and/or permeated the porous medium, they must be examined for the change regarding the reporting properties. In the flow-through method or in-situ method, the fluid with the particles and/or molecules flows past a sensor or a measuring device that detects both the reporting and the reference signal. This can be done, for example, by flowing through an optical flow cell in a spectrometer or fluorescence spectrometer.

In a batch method, one or more times a sample is taken and then examined. Sampling and transfer of liquid volumes into a measuring device in the sense of an ex-situ measurement. By sampling and a subsequent enrichment step, which aims to maintain the particles and/or molecules in a higher concentration with the aim of a stronger measurement signal of the reporting and reference functions, before the enriched sample is brought into the measuring device for measurement. Enrichment methods include centrifugation, ultracentrifugation or enrichment by evaporating part of the fluid.

Chemical precipitation reactions are also conceivable, in which the particles or molecules are precipitated out of the fluid and can then be examined in a highly enriched manner. Here, the application of the method is not limited to the flow application variant, in which the fluid with the particles is injected on one side and comes out on the other side. The method of pumping in and then sucking it out at the same point is also conceivable. In the case of the chromatographic analysis “last-in first-out”, one would expect that the particles injected last at the end would come out first.

Another variant is cross-diffusion: One analyzes (if necessary additionally) the diffusion across the current direction and obtains information about cross-diffusion constants, swirls etc. as well as channels of the cross-connection and their extent. In borehole experiments, the range is often used as the radial influence distance from the borehole.

For pumping tests e.g. the extension of the lowering funnel corresponds to the range of the pumping test. If the drilling depth (for imperfect wells) or the test interval (for packer tests) is taken into account for the range of groundwater thickness (for complete wells) or assuming negligible vertical flow components, the influence volume can be specified.

The spatial representativity that individual examination methods can achieve depends on the examination time and the size of the examination area, in addition to the hydraulic parameters. Methods that are usually carried out with short test times in a small examination area will have volumes of influence that are several orders of magnitude smaller than long-term tests with several observation points.

In a particularly advantageous variant of the method, a particle emits two signals during its examination after exiting the rocks, rock strata and/or porous materials or layers made of this porous material, which signals can be detected. First, a signal that signals the presence of the particle and that does not change due to the environmental conditions, and second, a signal that is also specific to the particle. But it is not emitted in the same way from its surroundings or the fluid and changes as a result of the environmental conditions if certain conditions have been reached during the flow and/or permeation (example: a temperature of at least 80° C.). The former signal has the function of a reference signal, the latter has the function of a reporting signal, which reports about the conditions experienced during the passage through the rock, the rock strata and/or porous materials or layers of this porous material in a memory function (memory effect) and reports about the properties and signals experienced. The advantage of the combination of reference and reporting signal is that it can be used to determine directly what percentage of the particles has seen said conditions.

Furthermore, the absolute concentration of the particles can be determined at any time, but also the relative concentration based on the concentration of the particles (number of particles per volume) based on the concentration when entering the porous medium). Specifically, the combination of reference and reporting signals can be implemented in very different ways.

An advantageous practical method is the combination of two molecules or molecular groups, which both emit fluorescent light at two different wavelengths when they are excited, for example, in the blue or ultraviolet spectral range. One of the two different molecules or molecular groups is stable against the possible environmental conditions in the porous medium, while the other is destroyed or irreversibly changed in its fluorescence properties when certain environmental conditions are reached. If a particle containing both molecules or molecular groups comes into detection after passing through the porous medium and after reaching the conditions necessary for the change, it is shown that the fluorescence of the former molecule is unchanged, but that of the latter is not. In this way, it is possible to quantitatively determine what percentage of all particles has reached the threshold condition for the change in the second molecule on the way through the porous medium.

The combination of reference property and reporting property for the marking function, within one and the same particle or molecule, can be realized in different ways. Particles carrying two functional groups that show fluorescence are used. This can e.g. happen by virtue of the fact that two corresponding molecules with the corresponding fluorescence properties are bound to the surface of the particle or are contained in its volume, or corresponding fluorescent groups of molecules are bound to the particle or on the surface of the particle. One group or one molecule is changed or destroyed under the threshold conditions, the other is not.

Thus, after reaching the corresponding threshold value, for example the temperature, or after exposure to the corresponding conditions, for example oxygen, ultraviolet radiation, radioactive radiation, the fluorescence of one molecule (=the reporting property) is changed, but that of the other (=the reference property) is not. Alternatively, we will use a molecule that carries two functional groups that exhibit fluorescence. One group is changed or destroyed under the threshold conditions, the other is not. Thus, after reaching the corresponding threshold value (for example the temperature) or after exposure to the corresponding conditions (oxygen, ultraviolet radiation, radioactive radiation etc.), the fluorescence of the molecule (=the reporting property) is changed, but the other (=the reference property) is not.

Both variants described above can also be used in combination. To this end, the first-mentioned molecule is used and incorporated into the alternatively mentioned particle or binds it (physically or chemically or physicochemically) to its surface. Use can also be made of particles or molecules that change their reporting properties almost immediately when the corresponding condition (threshold condition) is reached.

Use can be made of particles or molecules that continuously change their reporting properties as a function of the dose (extent and duration of exposure to, for example, oxygen or UV light or radioactive radiation). Such particles or molecules can then be used as a kind of microscopic dosimeter. Use can be made of particles or molecules that reversibly change their reporting properties with a certain decay time of the change after the end of exposure under the changing properties. Example: Temperatures of over 80° C. reversibly change the fluorescence of a particle (reporting property). This change decays with a decay time (half-life) of 183 seconds when the exposure at the temperatures mentioned ends. If you wait long enough, there is no change compared to the state before exposure. This variant has the advantage that it also provides information about how long the particles have been traveling from the time of exposure until they exit the porous medium.

By varying the flow velocity—for example via the applied pressure—both the percentage of particles exposed to the exposure and the time from exposure to arrival at the location of detection can be determined or estimated. A further embodiment consists in incorporating two different particles, one with the reference property and one with the reporting property, in a larger particle or to attach it to its surface (for example physisorbed, chemisorbed, or glued with an adhesive agent).

These can be optical properties, but also magnetic properties or magnetic resonance properties that disappear under the influence of oxygen for one type of nanoparticle, for example, but not for the other. Anaerobic situations in the rock can be directly detected in this application example. The combination of two or three of these methods allows comprehensive conclusions to be drawn about the percentage of particles that were exposed to the condition in question, the average dose (extent or concentration or intensity of exposure, integrated over time) and the average running times or dwell times of the fluid in the medium. The particle measurements can be carried out continuously in the flowing fluid stream by fluorescence spectroscopy with real-time evaluation of the spectra, where appropriate. An alarm—e.g. in the use as drinking water or as a warning of leakage—can then be given practically in real time, and you do not have to limit yourself to random samples, but can continuously observe. Chromatography is also possible in this way. This information can be correlated with the dwell time in the rock or the porous substance or the cave system.

Readout can be done by fluorescence etc. The particles can be measured in the by-flow or enriched by filtration. An interesting variant is the use of magnetic particles, which can be directed to specific locations by magnetic fields in a targeted way and which additionally allow the advantage of removal and enrichment by strong magnetic fields, so that the particles can be collected again for analysis after passage with magnetic help.

In an advantageous variant of the method, the particles that are dispersed in a fluid, for example liquid, are treated by means of ultrasound before the fluid is injected with the particles for the passage through the porous medium. In this way, particles that have partially coagulated to form aggregates are dispersed again into individual particles.

As described above, the reporting function can be any property or combination of different properties, which is changed or not change due to the environmental conditions experienced when flowing through the porous medium. A property that is very suitable for the detection is the change in the chemical or biochemical or spectroscopic property or other properties by splitting off a molecular group. If it is simply a matter of splitting off hydrogen in the form of H or H⁺, however, this reaction is usually reversible: H⁺ ions are ubiquitous in the aqueous medium. However, if the functional units are more complex, the split-off reaction will remain irreversible even if the reaction conditions change again during the passage and the conditions for the reverse reaction to take place exist: The group that has been split off has long since diffused away or has been moved to another location with the flow and is no longer available for the reverse reaction. If the particle splits off such a group due to environmental conditions, then this chemical group or this molecular fragment or ion never returns to the starting point (for reasons of probability) and the reverse reaction never takes place again, even if the conditions for this are given again later. As the group or species to be split off, it is important to use a group or species that is not available anyway (“ubiquitous”) in the fluid flowing or pumped through the porous medium.

The group to be split off can be split off as a radical, as a cation, as an anion, as a molecule or molecule fragment or even as a particle.

The split-off may take place in the form of a chemical reaction in the form of a molecular predetermined breaking point, which is broken up by light adsorption, UV radiation, radioactive radiation, pH value, oxidative influence or ionic strength or by specific or unspecific chemical or biochemical reactions by the presence of specific molecules or by oxygen, by ozone, by oxidizing agents.

In addition to the split-off, it is also possible to block or protect a specific molecular functional group or unit. Furthermore, an addition reaction, complexation, etc. can also change the properties of a group in such a way that the reporting property changes.

An important variant is the detection of certain ions via complex formation, which often goes hand in hand with a significant color change, as well as other color reactions, which makes it possible to very simply spectroscopically detect the reaction and thus the presence of the species triggering the reaction (e.g. a metal ion, metal or metal oxide). Examples are iron detection with thioglycolic acid or with hexacyanoferrates or with thiocyanates.

A completely different possibility is the precipitation of particles, for example from a certain ionic strength onward. For example, five different particles are sent into the porous medium, all of which are roughly the same size, but are marked with five different detection functions, and differ in the ionic strength or the pH value at which they can no longer be dispersed in the fluid (e.g. in the aqueous medium), but are precipitated. If all five are simultaneously injected into the porous medium at the same location, but only two or three of the species come out at the other end, it can be concluded that the others have been precipitated, which provides direct conclusions about the ionic strength or the pH value experienced.

Magnetic particles are of great interest for the method according to the invention in various respects: (1) Firstly, the magnetic property of the particles can be used very well as a reference or recognition property—at least if magnetic nanoparticles cannot be found ubiquitously in the porous medium. (2) Furthermore, the magnetic property allows a simply possibility of enrichment after passage through the porous medium. (3) In addition, the magnetic property can alternatively also be used as a reporting property: The magnetic property can be destroyed by a variety of chemical reactions with the particle that was still magnetic before the reaction.

An interesting approach is the systematic degluing or detachment when the threshold condition is reached. A central particle contains the property with which it is recognized in itself. Other species, for example other (e.g. smaller) particles, are “glued” to its surface via a specific interaction or an “adhesive” in the broadest sense. These particles contain the reporting property. One example is the use of microparticles with nanoparticles glued to their surface. The adhesive can be a wax or a polymer that softens or melts at a certain temperature, so that the adhesive loosens and the small ones separate from the large particles. If, for example, the large particles are enriched by centrifugation after flowing through the porous medium, they lack the reporting property. A particularly advantageous configuration of this variant is the use of magnetic particles either for the glued-on particles or for the central particle. For example, if the central particle is ferromagnetic and the glued-on particles are fluorescent, the central particles can be magnetically enriched after flowing through the medium. If they no longer show fluorescence, the adhesive bond between the central particle and the glued-on particles has broken off on the way through the porous medium: The softening temperature of the glue was obviously exceeded. Accordingly, bonds and adhesive forces between the particles can also be used, which depend on the ionic strength, the pH value, the chemical and biochemical conditions or the oxygen attack.

The following different variants can inter alia be used for the split-off of chemical groups as well as for selective degluing when certain conditions are experienced: (1) split-off by light (ultraviolet, infrared, visible light), (2) split-off by other electromagnetic radiation, e.g. by microwaves, (3) split-off by other radiation, in particular by radioactive radiation (with the option of use as the smallest dosimeter), (4) cleavage by exceeding or falling below a certain pH value, (5) cleavage by the action of water “humidity-induced cleavage) and (6) catalytic cleavage or photo-catalytic cleavage (e.g. in the presence of titanium dioxide particles and light at the same time).

The latter variant also shows an example of a further variant of the method, namely the possibility of checking the simultaneous presence of more than one property (property coincidence check): the simultaneous presence of light and photocatalyst, or in another example: the simultaneous presence of oxygen and a minimum temperature for the oxidation reaction.

The method according to the invention is consequently used in the quantification of rocks, rock strata and/or porous materials or layers made of this porous material, in the geological analysis of rocks, rock strata and/or porous materials or layers made of this porous material, in hydrology, water exploration, deposit exploration, deposit monitoring, fracking, geothermal energy, leakage monitoring, but also in the monitoring of chemical, biological and/or biotechnological reactors, or in medical in-vivo methods.

Technically, the use of particulate moisture sensors, for example, should play an important role. The detection of oil in rock (change in the reporting properties due to the presence of (traces of) oil is technically highly relevant. The same applies to the detection of heavy metals. Here, the porous medium could, for example, be the soil in the surrounding of a landfill or in the catchment area of a drinking water reservoir.

A potential application is also to ensure the sealing or the location of leakages in landfills, hazardous waste landfills and other deposit areas. If such tracer particles are introduced at certain locations in the stored goods, then when such particles are found, e.g. in the groundwater or landfill drainage water, the locations where the leak occurred can precisely be determined. This also applies to the long-term monitoring of leakage from car washes, radioactive or chemically contaminated pipe systems or cisterns. Monitoring can be extremely cost-effective and continuous at the same time.

The method is suitable for the non-destructive internal exploration of porous media and their property control (to ensure quality and properties during production) and for the exploration of deposits of oil, natural gas, mineral resources, geothermal energy, porosity of rocks and the inexpensive, non-destructive exploration of rock strata, for example in the area of tunnel construction, to name just a few examples.

However, researching the parameters inside rock strata and soil formations is a central problem, for example, with: geothermal energy, extraction of mineral resources and research into their deposits, water routes in the area of landfills, final deposits of toxic and radioactive waste or fracking. Hydraulic fracturing or briefly fracking (from English “to fracture” to tear open; German also hydraulic fracturing, hydraulic breaking, hydraulic crack generation or hydraulic stimulation) is a method for the generation, expansion and stabilization of cracks in the rock of a deposit in the deep underground with the aim of increasing the permeability of the deposit rocks. As a result, gases or liquids contained therein can flow more easily and more reliably to the borehole and be extracted. In an important application variant, a gaseous fluid is used to generate a gas flow and the particles and/or the particle mixture is added to the gas flow. This is very relevant to the application, for example, in the exploration of natural gas deposits and their flow through the rock.

The method according to the invention is also particularly suitable for use in chemical and biological/biotechnological reactors and on water tanks, water reservoirs and water pipe systems. In these applications, the inside of the chemical and biological/biotechnological reactors as well as the inside of the water tanks, water reservoirs and water pipe systems is to be understood as the “porous medium”.

For example, checking whether a maximum temperature is exceeded in chemical reactors can be mentioned as a special use. This is of great interest, for example, in polymerization reactions, where thermal decomposition products and carbon are formed when certain temperature values are exceeded, which adversely affects the color of the resulting polymer—and also on the electrical insulation properties. The method according to the invention can be used to determine whether and to what extent such temperature overshoots occurred. The temperature values that were exceeded can be determined with the tracer particles with the corresponding threshold value in the reporting function, as well as the extent of the exceedance. Specifically, what percentage of the particles experienced the exceedance after leaving the reactor based on their reporting function.

The examination can also be carried out by spectroscopy of the particles directly in the corresponding reaction product leaving the reactor or by subsequent extraction of the particles from the reaction product leaving the reactor, for example by ultracentrifugation or by extraction of ferromagnetic particles using a magnetic field.

Another possibility is that the method according to the invention is used in medical in-vivo methods. The fluid with the particles with the reference function and the at least one reporting function is injected into a human or animal body, for example to carry out diagnostic procedures on the bloodstream, lymphatic system, urinary system, digestive tract, lungs and airways, nose and sinuses.

The present invention accordingly also relates to a fluid with at least one particle or a mixture of particles for use in a medical in-vivo method, the particles having a reference function and at least one reporting function for detecting physical, chemical or biochemical parameters.

1. A method for quantifying a porous medium with at least one particle or a mixture of particles, wherein the at least one particle or the mixture of particles have a reference function and at least one reporting function for recording physical, chemical or biochemical parameters of the porous medium, said method comprising the following steps:
introducing the at least one particle or the mixture of particles into a fluid,
letting the fluid with the at least one particle or the mixture of particles flow through and/or permeate the porous medium, wherein the at least one reporting function of the at least one particle or the mixture of particles changes when a threshold value of the parameters to be recorded is exceeded or fallen below, while the reference function of the at least one particle or the mixture of particles remains unchanged, and
after exiting the porous medium, at least one subsequent analysis of the at least one particle or the mixture of particles for a physically, chemically or biochemically changed reporting function and the reference function of the at least one particle or the mixture of particles, wherein the reference function serves to recognize the at least one particle or the mixture of particles.

2. The method according to claim 1, wherein the at least one particle or the mixture of particles have a diameter ranging from 100 μm to 0.5 nm.

3. The method according to claim 1, wherein the base body of the at least one particle or the mixture of particles consists of silver, gold, copper or other metals, silicon dioxide, polystyrene, olefins, wax or a mixture thereof.

4. The method according to claim 1, wherein the at least one reporting function is contained in the at least one particle or the mixture of particles and/or on a surface of the at least one particle or the mixture of particles, comprising at least one fluorescence marker, a luminescence marker, a marker for the plasmonic property, a pH value indicator, a temperature indicator, a radiation indicator alone or in combination with one another.

5. The method according to claim 1, wherein the change in the at least one reporting function is irreversible.

6. The method according to claim 1, wherein the change in the at least one reporting function increases continuously with a dose of radiation (radiation exposure) or oxidative stress experienced.

7. The method according to claim 1, wherein the reference function of the at least one particle or the mixture of particles is in the form of a geometric and/or haptic detection site, or in the form of a fluorescence marker, a luminescence marker or a marker for the plasmonic property.

8. The method according to claim 1, wherein the at least one particle or the mixture of particles further have an additional function that allows determining the residence time of the at least one particle or the mixture of particles in the porous medium on the basis of a time-dependent disintegration or a time-dependent change in a property.

9. The method according to claim 1, wherein the at least one particle or the mixture of particles further have a magnetic additional function.

10. The method according to claim 1, wherein the at least one particle or the mixture of particles have a further reporting function that changes when a threshold value of a second parameter to be recorded, which is different from a first parameter, is exceeded or fallen below the second parameter.

11. The method according to claim 1, wherein the porous medium is a liquid- or gas-filled space.

12. The method according to claim 1, wherein the porous medium comprises rocks, rock strata and/or a porous material or layers made of this porous material.

13. The method according to claim 1, wherein the analysis of the at least one particle or the mixture of particles is carried out by optical spectroscopy, IR spectroscopy, plasmonic resonance, microscopy, dosimetry, nuclear magnetic resonance, electron spin resonance, ENDOR, fluorescence spectroscopy, single-molecule fluorescence spectroscopy, atomic fluorescence spectroscopy, luminescence spectroscopy, photoluminescence spectroscopy, chromatography, gas chromatography, liquid chromatography and/or high-performance liquid chromatography (HPLC), or by means of a follow-up reaction which facilitates the detection of the change in the at least one reporting function.

14. The method according to claim 1 for quantifying rocks, rock strata and/or porous materials or layers of this porous material, in the geological analysis of rocks, rock strata and/or porous materials or layers of this porous material, in hydrology, water exploration, deposit exploration, deposit monitoring, fracking, geothermal energy, leakage monitoring, monitoring of chemical, biological and/or biotechnological reactors, of water tanks, water reservoirs and water supply systems, or in medical in-vivo methods.

15. A fluid with at least one particle or a mixture of particles for use in a medical in-vivo method, wherein the at least one particle or the mixture of particles have a reference function and at least one reporting function for recording physical, chemical or biochemical parameters.

16. The method according to claim 1, wherein the at least one particle or the mixture of particles have a diameter ranging from 10 μm to 5 nm.

17. The method according to claim 1, wherein the at least one particle or the mixture of particles have a diameter ranging from 5 μm to 50 nm.
